(19) United States
(12) Patent Application Publication
(10) Pub. No.: US 2025/0223250 A1
(43) Pub. Date: Jul. 10, 2025

(54) METHODS FOR OXIDATION OF METHANE TO METHANOL, SYSTEMS FOR OXIDATION OF METHANE TO METHANOL, AND DEVICES FOR OXIDATION OF METHANE TO METHANOL

(71) Applicant: Board of Supervisors of Louisiana State University and Agricultural and Mechanical College, Baton Rouge, LA (US)

(72) Inventors: Kerry Matthew Dooley, Baton Rouge, LA (US); James A. Dorman, Baton Rouge, LA (US)

(21) Appl. No.: 18/850,661

(22) PCT Filed: Apr. 7, 2023
(86) PCT No.: PCT/US2023/065492

§ 371 Date: Sep. 25, 2024

Related U.S. Application Data
(60) Provisional application No. 63/328,797, filed on Apr. 8, 2022.

(51) Int. Cl. C07C 29/50; B01J 12/00; B01J 19/00; B01J 19/24; C07C 31/04; C22C 19/05; C23C 8/20

(52) U.S. Cl. CPC C07C 29/50; B01J 12/00; B01J 19/0093; B01J 19/2415; C07C 31/04; C22C 19/055; C23C 8/20; B01J 2219/00822; B01J 2219/00837

(57) ABSTRACT

The present disclosure provides for systems and methods for selectively oxidizing methane to methanol, devices including reactors, reactors for selectively oxidizing methane to methanol, method of making coated reactor tubes of reactors, and the like. In an aspect, the system can have the characteristic of a per pass methanol yield of greater than 8% and selectivities of greater than 70% for methane to methanol.

### CROSS-REFERENCE TO RELATED APPLICATIONS

This application claims the benefit of and priority to U.S. Provisional Application Ser. No. 63/328,797, having the title “Selective Non-Catalytic Direct Partial Oxidation of Methane to Methanol in a Wall-Coated Microreactor” filed on Apr. 8, 2022, the disclosure of which is incorporated herein in by reference in its entirety.

### BACKGROUND

Large amounts of methane in remote locations are currently flared due to poor transportation economics and high costs of conversion to a more easily transportable fuel. While gaseous methane must be pipelined, methane can be converted into liquid methanol for easier transportation via ship, barge, rail, or truck. Thus, there is a need to convert methane to methanol that is advantageous to current technologies.

### SUMMARY

Embodiments of the present disclosure provide for systems and methods for selectively oxidizing methane to methanol, devices including reactors, reactors for selectively oxidizing methane to methanol, method of making coated reactor tubes of reactors, and the like.

An embodiment of the present disclosure provides for a device comprising: a reactor having a plurality of reactor tubes, where walls of the reactor tubes are coated with an inert coating, wherein the reactor tube is made of a nickel-based metal alloy. In an aspect, the nickel-based metal alloy has a composition comprising: a weight percent of about 58 to 66% Ni, a weight percent of about 20 to 23% Cr, a weight percent of about 8 to 10% Mo, a weight percent of about 0.01 to 5% Fe, a weight percent of about 3 to 4% Nb and Ta, and a weight percent of about of about 0.01 to 1% Co. The inert coating can be a carbon/carbide layer and can be about 50 to 200 microns thick.

An embodiment of the present disclosure provides for a system for selectively oxidizing methane to methanol, comprising: a reactor of any one of claims 1 to 8, wherein the system has the characteristic of a per pass methanol yield of greater than 8% and selectivities of greater than 70% for methane to methanol. In an aspect, the system can have a total reactor pressure of about 70 to 100 bar during operation, an outer wall temperature of about 380-440° C. in a hot zone of the reactor tube, an outer wall temperature of about 200-250° C. in a quench zone of the reactor tube, and wherein a methane and air mixture having a methane/air molar ratio 2.5 to 3.3 is introduced to the reactor tubes to a pressure of about 70 to 90 bar and the methane and air mixtures has a residence time in the reactor tubes of about 0.6 to 1 min or about 0.8 min. A temperature of each zone of the reactor tubes can be controlled using a heat bath, a heat jacket, a chiller, or a combination of these. The system further comprises a gas flow system in gaseous communication with the entrance and the exit of the reactor tubes, where the gas flow system is configured to flow a methane and air mixture into the entrance of the reactor tubes, where the ratio of the methane to air mixture is controlled using mass flow controllers, where a backpressure regulator is in gaseous communication with the exit of the reactor tubes, where the backpressure regulator is configured to control the pressure in the reactor, where the gas flow system is configured to flow the methane and air mixtures into the reactor tubes to reach a pressure of about 70 to 90 bar, where the reactor is configured to control the temperature of the outer wall of the hot zone to be about 380-440° C., where the reactor is configured to control the temperature of the outer wall of the quench zone to be about 200-250° C., where the gas flow system is configured to receive the methanol from the exit of the reactor tubes resulting from the oxidization of the methane to methanol.

The present disclosure provides for a method of selectively oxidizing methane to methanol, comprising: introducing methane and air to the reactor as describe above or herein, wherein the methane and air are introduced via the entrance of the reactor tubes at a ratio of about 2.5 to 3.3 until a pressure of 60 to 100 bar is reached; regulating a temperature of the hot zone of the reactor tubes to be about 380-440° C.; forming a mixture comprising methanol; regulating a temperature of the quench zone of the reactor tubes to be about 200-250° C.; and collecting the mixture comprising the methanol. The method has a per pass methanol yield of greater than 8%. The method has a selectivities of greater than 70% for methane to methanol.

The present disclosure provides for a method of coating a reactor wall, comprising: introducing propane to a reactor tube of a reactor, wherein the reactor tube is made of a nickel-based metal alloy; decomposing the propane at a temperature of about 650 to 750° C., and forming an inert coating on the inside walls of the reactor tube.

Other methods, system, devices, and features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 illustrate a tube core of advanced oxidation microreactor. There are 414 tubes, 1 m long, 2.41 mm outside diameter, 1 mm inside tube diameter.

FIG. 2 illustrates a microreactor system.

FIG. 3A illustrates an uncoated I625 coupon. Coupons are approximately 76×6.4×1.6 mm.

FIG. 3B illustrates a carbided I625 coupon from graphite-salt flux synthesis, front and back.

FIG. 3C illustrates a carbided I625 coupon from propane-based synthesis, front and back.

FIG. 4 illustrates a TGA/DSC of scraped overlayer from a carbided I625 coupon. The vertical lines in the wt % curve mark temperature holds at 100, 550 and 850° C.

FIGS. 5A and 5B illustrates TGA/DSC of scraped overlayers from carbided I625 coupons and standards (to 400° C.)

FIG. 6 illustrates a microreactor carbiding tests 1-3. For test 1 (a), the temperature of the reactor was increased gradually over 3 d from 500 to 690° C. For tests 2 (b) and 3 (c), the temperature of the reactor was 690° C. In all cases the experiment was halted before the tubes were blocked.

FIGS. 7A and 7B illustrates the results of DMTM Experiments producing significant methanol. The X axis labels are temperature (° C.), pressure (bar), methane:air molar ratio, and residence time(s).

FIG. 8 illustrates experimental selectivities for CH₃OH as a function of CH₄ conversion, adapted with permission from Rasmussen and Glarborg [30]. Copyright 2008 American Chemical Society. Additions from the literature of 2008-2015.

FIG. 9 illustrates a XRD of the carbide layer.

FIG. 10 illustrate various SEM images of the carbide layer.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method may be carried out in the order of events recited or in any other order that is logically possible.

Embodiments of the present disclosure will employ, unless otherwise indicated, techniques of chemistry, material science, and the like, which are within the skill of the art. Such techniques are explained fully in the literature.

Prior to describing the various embodiments, the following definitions are provided and should be used unless otherwise indicated.

Unless otherwise defined, all technical and scientific terms used herein have the same meaning as commonly understood by one of ordinary skill in the art of microbiology, molecular biology, medicinal chemistry, and/or organic chemistry. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DISCUSSION

Embodiments of the present disclosure provide for systems and methods for selectively oxidizing methane to methanol, devices including reactors, reactors for selectively oxidizing methane to methanol, method of making coated reactor tubes of reactors, and the like. Aspects of the present disclosure are advantageous in that the reaction of methane to form methanol can be performed in an economically efficient manner. As a result, aspects of the present disclosure can be used in situations where methane is typically burnt off and can now produce methanol and ultimately reduce CO₂ emissions caused by methane burn-off. In an aspect, the reactor can include reactor tubes that have walls that are inert, which reduces competing reactions that reduce the conversion rate of methane to methanol. Thus, the reactor tubes having the coating increase specific conversion of methane to methanol. Additional advantages and features are provided herein and in Example 1.

In an aspect, the present disclosure provides for systems and methods for selectively oxidizing methane to methanol (See FIG. 2 and Example 1 discussion). The systems and methods have the characteristic of a per pass methanol yield of greater than 8% (e.g., 8 to 20%, 8 to 15%, or 8 to 10%) and selectivities of greater than 70% (e.g., 70 to 90%, 70 to 80%) for methane to methanol conversion.

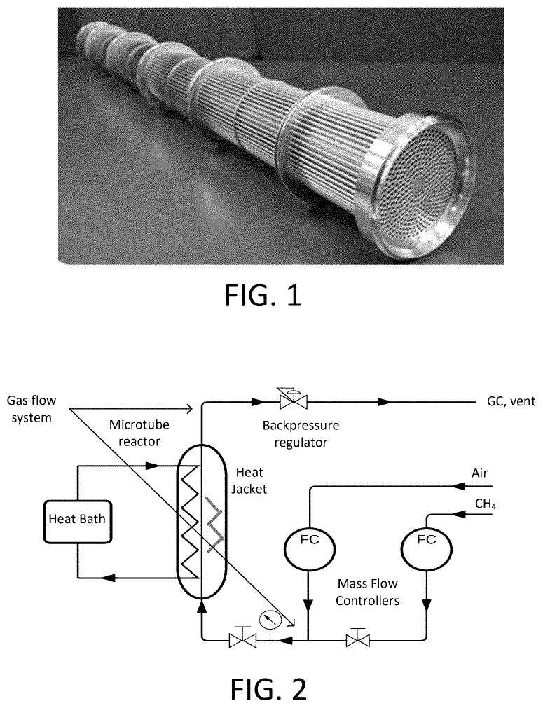

The selective oxidation of methane to methanol can be accomplished using devices or systems that include a reactor having a plurality of reactor tubes (e.g., see FIG. 1). The walls of the reactor tubes are coated with an inert coating. The reactor tube can be made of a nickel-based metal alloy (e.g., Inconel® 625). In an aspect, the nickel-based metal alloy can have a composition comprising: a weight percent of about 58 to 66%, about 58 to 60%, or about 60% Ni, a weight percent of about 20 to 23% or about 22% Cr, a weight percent of about 8 to 10% or about 8% Mo, a weight percent of 0 to 5%, about 0.0.1 to 5%, or about 4% Fe, a weight percent of about 3 to 4% or about 3% Nb and Ta, and a weight percent of about of about 0 to 1%, about 0.01 to 1%, or about 0.05% Co, where other components (e.g., elements such as Al, Ti, C, Mn, Si, P and S) can be present are less than 1% or less than 0.1%. Each reactor tube can have an inside diameter of about 0.9 to 1.1 millimeters or about 1 millimeter. The reactor can have tens to thousands of reactor tubes (e.g., 10 to 10,000, 100 to 5000, 500 to 2500, or about 1000 to 2000). The number of reactor tubes can be a function of controlling the temperature of the various zones of the reactor tubes so that the reactor operates efficiently to specifically convert methane to methanol. The length of the reactor tubes can be varied based on the desired configuration can be in the cm to meter range. In an aspect, the length of the reactor tubes can be about 5 cm to 10 m, about 5 cm to 5 m, about 5 cm to 1 m, about 5 cm to 500 cm, about 5 cm to 250 cm, about 5 cm to 100 cm, or about 5 cm to 50 cm.

The inert coating disposed on the inside walls of the reactor tubes can be a carbon/carbide layer. The carbon/carbide layer comprises graphite oxide, semi-amorphous carbon, graphitic carbon, chromium carbide phase Cr₇C₃ and chromium carbide phase Cr₂₃C₆. The carbon/carbide layer can include sublayers of graphite oxide, semi-amorphous carbon, graphitic carbon, chromium carbide phase Cr₇C₃ and chromium carbide phase Cr₂₃C₆. The amount of each of graphite oxide, semi-amorphous carbon, graphitic carbon, chromium carbide phase Cr₇C₃ and chromium carbide phase Cr₂₃C₆ can vary but within amounts that the carbon/carbide layer retains the characteristic of being inert in the conversion of methane to methanol under the conditions described herein. The inert coating can be about 50 to 200 microns or about 100 to 150 micron thick.

Each reactor tube can have multiple zones along the length where mixing, reacting, and quenching of the reaction occur. In other words, the methane and air mixture (mixture of both methane and air) is introduced to the entrance of the reactor tube and proceeds through the reactor tube and reacts, is quenched, and then the products flow out of the reactor tube. The temperature in each zone can be controlled and regulated to efficiently mix, react, and quench. Each reactor tube can have a mixing zone, a hot zone, and a quench zone. The mixing zone is the portion of the reactor tube where the methane and air are mixed so that the reaction can proceed efficiently, the hot zone is the portion of the reactor tube where the conversion of methane to methanol occurs, and the quench zone is where the reaction is cooled to reduce or eliminate unwanted reactions so as to maximize the specific conversion of methane to methanol. Each reactor tube has a length, where the mixing zone is the first portion of the length of the reactor tube, the hot zone is a second portion of the length of the reactor tube, and the quench zone is a third portion of the length of the reactor tube. The second portion of the reactor tube is between the first portion of the reactor tube and the third portion of the reactor tube. The first portion is adjacent an entrance to the reactor tube and the third portion is adjacent the exit of the reactor tube. The first portion is about 8 to 50% or 8 to 30% of the length, wherein the second portion is about 7 to 20%, about 7 to 15%, or about 8% of the length, and the third portion is about 2 to 50%, 2 to 20%, or 2 to 10% of the length.

The reactor including the plurality of reactor tubes can be in fluidic communication with a heat bath and a chiller, and adjacent a heat jacket. The temperature controlling and regulating devices can be configured to control and regulate the temperature of each zone independently. For example, the heat bath and heat jacket can control and regulate the temperature of the hot zone (e.g., about 380-440° C.), while the chiller can control and regulate the temperature of the quenching zone (e.g., about 200-250° C.). Also, one or more other temperature control devices can be used to control and regulate the temperature of other areas of the device such as the tubes into and out of the reactor, methane temperature and air temperature.

The devices and systems can include the reactor and they can also include a gas flow system the controls the introduction of the methane and air, introduction of the methane and air mixture to the reactor (e.g., reactor tubes), receive the products of the reaction from the reactor, control the pressure in the reactor tubes, and capture the products. The gas flow system can be in gaseous communication with the entrance and the exit of the reactor tubes so that the methane and air mixture can flow into the reactor and then the products can exit the reactor. The ratio of the methane to air mixture can be controlled using mass flow controllers. A backpressure regulator is in gaseous communication with the exit of the reactor tubes and can function to control the pressure of the gasses in the reactor. In other words, the gas flow system is configured to flow the methane and air mixture into the reactor tubes and the backpressure regulator stops or reduces the flow until a pressure of about 60 to 100 bar or about 80 bar is reached within the reactor tubes. Once the reactor tube reaches 80 bar, the reaction can be initiated. The system and reactor are configured to control the temperature of the outer wall of the hot zone to be about 380-440° C. The system and reactor are configured to control the temperature of the outer wall of the quench zone to be about 200-250° C. After a period of time, the gas flow system is configured to receive the methanol from the exit of the reactor tubes resulting from the oxidization of the methane to methanol. The temperature of each zone can be measured using an independent thermocouple or similar temperature measurement device.

The gas flow system can include tubing (e.g., for gas and fluid), flow controllers, temperature measuring devices, valves, pumps, and other components found in gas flow systems. FIG. 2 illustrates some of the components used in the gas flow system but other components can be used as well to operate the system.

The present disclosure provides for methods of selectively oxidizing methane to methanol. In an aspect, the method can include introducing methane and air to a reactor or system such as those described herein. The methane and air mixture is introduced via the entrance of the reactor tubes at a ratio of about 2.5 to 3.3 or about 2.9 until a pressure of 80 bar is reached, and at this point the reaction can be initiated. The temperature in the hot zone (e.g., about 380-440° C.) and the quenching zone (e.g., about 200-250° C.) can be regulated to produce methanol (e.g., a selectivities of greater than 70% for methane to methanol). The methanol can then be collected as it exits the reactor. The method has a per pass methanol yield of greater than 8% (e.g., 8 to 20%, 8 to 15%, or 8 to 10%) with selectivities of greater than 70% (e.g., 70 to 90%, 70 to 80%) for methane to methanol conversion.

The present disclosure also provides for method of coating a reactor wall, for example the reactor tubes described herein. The method includes introducing propane to a reactor tube of a reactor. The reactor tube is made of a nickel-based metal alloy, such as those described above and herein. The propane is decomposed at a temperature of about 650 to 750 or about 700° C. to form an inert coating on the inside walls of the reactor tube. The inert layer is the same as already described above and also described in Example 1.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### Example 1

Large amounts of methane in remote locations are currently flared due to poor transportation economics and high costs of conversion to a more easily transportable fuel. While gaseous methane must be pipelined, methane can be converted into liquid methanol for easier transportation via ship, barge, rail or truck. Currently, methane is converted to methanol commercially via a two-step process. First, the methane is steam-reformed to synthesis gas (CO and H₂), which then undergoes a high-pressure catalytic conversion to methanol [1].

A non-catalytic, direct methane to methanol (DMTM) continuous process has existed for some time. It is characterized by much higher per pass conversions than competing direct catalytic processes, and a computed lower cost than the steam reforming-high pressure CO hydrogenation route, if the per pass methanol yield can be brought to >8% at 70+% selectivity (economic studies summarized by Turan et al.) [2]. DMTM has been demonstrated by many research groups, some getting per pass methanol yields>8%, but not always reproducibly. In the 80-90's, several groups using homogeneous DMTM processes, including Gesser et al. [3, 4] and Knopf and Dooley [5] reached this target. More recently other groups have come reasonably close to reproducing the older results [6, 7]. However, all of these works relied upon reactors with exotic and/or fragile wall materials, hot-wire filament or plasma radical initiation, sensitizers (chemical radical initiators), sophisticated mixing designs, or some combination of these, making scale-up cost-prohibitive.

It has been shown that for dielectric barrier discharge (DBD)-based processes total liquid product yields (mostly CH₃OH, HCHO, HCOOH, but with several other oxygenates) near 20% are possible [8]. A non-thermal plasma such as DBD is a reliable albeit expensive way (the energy efficiency could be as low as 1%) [9] to generate greater concentrations of free radical species at relatively low temperatures where total combustion is minimized. By combining a DBD reactor and a suitable catalyst, the CH₃OH selectivity has been increased from older work, where it did not exceed 22% [10]. For example, with Ni/Al₂O₃ at <0.4 s residence time, ˜50% CH₃OH was obtained, but at low CH₄ conversion. Higher conversions at longer residence times give higher CH₃OH yields but lower selectivity (e.g., Fe/Al₂O₃, 36% CH₃OH selectivity at 13% conversion) [12].

In addition to increased yields to methanol, the DMTM process could save ˜$500,000/yr for one remote wellhead installation and reduce its carbon footprint by 104 metric tons of CO₂, because methanol is also added to pipeline gas at remote locations to prevent hydrate formation in long pipelines [13-22]. Methanol is also used as a feedstock for many chemical processes such as acetic acid, formaldehyde, acrylates, and ethers; it is a basic building block of the chemical industry. Finally, it is also being used now in wastewater treatment facilities worldwide as a denitrification agent (converts excess nitrate to N₂). Excess nitrate causes algal bloom in waters. Methanol is the most common organic compound in denitrification, added in nearly 200 wastewater treatment facilities in the U.S. [23]. Usage rates vary widely with plant size and nitrate loadings, in the 25,000-120,000 gal/yr range [24]. In remote locations, it may prove easier to make this methanol onsite rather than truck it to the facility.

The economic tipping point for DMTM using stranded natural gas is the production cost of methanol via the standard (steam reforming, CO hydrogenation) route, which is roughly $1.60-1.90 per gasoline gallon equivalent for natural gas priced at $6-7 per MMBtu [25]. While we cannot assess the wildly variable costs of methanol production from stranded gas at this time, the supply of such gas is immense, at least 6,000 Tcf, compared to U.S. annual production of ˜33 Tcf/yr [17].

There are five keys to making the DMTM process more selective to methanol, the first being elevated pressure. The rates of radical termination reactions, which lead preferentially to methanol at low temperature, increase linearly with fluid-phase viscosity; this is but one reason why methanol selectivity increases with total pressure [28]. Another reason is the approximate second-order nature of methanol production (vs. first order for CO production) [29]. The selectivity to methanol typically increases rapidly up to ˜80 bar, then there is only a modest further yield increase at up to 200 bar [5, 29-33]. This dependence is also due to a switch in the mechanism from a single radical chain carrier process to a chain branching process [28]. This can be seen from the reactions below:

CH₄+O₂→CH₃·+HO₂·(Initiation)

CH₃·+O₂⇄CH₃OO·(Initiation)

CH₃OO·+CH₄(CH₃OH,CH₂O,HOOH)→CH₃OOH⇄CH₃O·+OH·(Branching)

The gas phase reaction chemistry here is complex, typically involving >40 species and >300 reactions [28-30, 32].

The second key is rapid mixing of the reactants, which can be enhanced by decreasing the residence time (higher velocity). Kinetics simulations have shown that a narrower high-temperature reaction zone resulting from higher velocity followed by rapid quenching can enhance the yields [5, 28-30].

The third key is to employ inert (to O₂ adsorption) solid surfaces for tube walls, because in real reactors the homogeneous process also includes heterogeneous (wall) reactions [28, 29, 34]. If the residence time exceeds the characteristic diffusion times (to the walls) of reacting species, then wall effects become appreciable [33]. Therefore reactor materials of construction greatly affect observed yields [32, 35]. All conventional stainless steels catalyze the complete combustion of methane [29, 36]. Some ways to effect passivation include using glass/quartz reactors, which are fragile, or more expensive inert surfaces such as sapphire or alundum, or by passivating the steel with a less active (for oxidation) coating. The passivated layer must be inert not only to methane combustion, but to its own oxidation as well, as the metal oxides can also combust methane.

Using carbon for passivation of Ni—Cr alloys has been studied for many years. Both Cr₃C₂ and Cr₇C₃ layers on stainless steels have been characterized [37]. More recent studies were able to synthesize and characterize the entire range of Cr carbides, including Cr₂₃C₆, Cr₂C and CrC [38]. In ethane pyrolysis furnaces coarse Cr carbide deposits form on Ni—Cr alloys [39]. It was also found that simple high-temperature (>625° C.) hydrocarbon decomposition gives durable carbide coatings on Ni [40]. These carbide layers have been characterized in the literature, but with conflicting results. One study found that carbide precipitated on Ni—Cr alloys increased brittle fracture, hardness, and ductility simultaneously [39]. Another study found that Cr carbide layers exhibited high melting points, extreme hardness, and good corrosion resistance [41]. Therefore, it might be concluded that extensive carbiding would be detrimental to long-term use of the metal, but thin carbide layers could be tolerated.

The fourth key is the methane to air ratio. The selectivity to methanol decreases monotonically with increasing oxygen concentration, but yield goes through a maximum. Many studies find a maximum yield at ˜2.5% oxygen [42]. The fifth and final key is that rapid quenching of the reaction is needed, because the product methanol is more reactive than methane itself, and because chain branching can grow exponentially.

We show here that the limitations on past DMTM processes can be partly overcome by combining microreactor technology with coating procedures to passivate microreactor inner tubes. We tested different coating procedures on several metals/alloys, finding one procedure and one alloy (Inconel I625) that could be carbided extensively. We then employed an integrated mixer-reactor-heat exchanger microtube scalable system for the non-catalytic direct partial oxidation of methane to methanol. With this system the five key parameters can be fine-tuned to selectively produce methanol at per pass yields>8% and selectivities >70%. Therefore the yield of the DMTM process can be brought closer to that of the methane plasma (DBD) oxidation process.

### 1. Materials and Methods

### 1.1 Passivation of Reactor Tubes.

This work uses mixed carbides as passivating layers. Sulfide layers made from elemental sulfur or the decomposition of dimethyl sulfide or dimethylsulfoxide were also tested but were found to be inferior to the carbide layers after oxidative stability testing. Several steels in the form of corrosion coupons (Metal Samples, 12.7×76×1.6 mm, cut in half lengthwise) were tested for forming carbide and sulfide layers in reactive flows. These metals were C1010 (mild steel), type 304L (low Ni, Cr content), type 904L (higher Ni, Cr content), type Inconel® |625 (highest Ni, Cr content of a commercial alloy), Nichrome (80 wt % Ni, 20 wt % Cr) and pure Ni itself. In the end I625 was chosen for further study, because it gave coatings of reasonable uniformity and integrity (unlike Nichrome or pure Ni), and without obvious corrosion of the underlying metal (unlike C1010). Type 904L was next best.

### 1.2. Coating Analysis.

To identify the chief component of the carbided coatings (Fe, Ni or Cr carbides, or forms of carbon), a series of standards and samples of the layers themselves were reacted in methane-air flow at typical reactor temperatures in a TGA/DSC (Perkin-Elmer STA-6000). The Cr carbide standard was from Strem (>97.5%). The Fe carbide was synthesized by reacting hematite and pure CO for 1 h at 500° C. [43]. Ni carbide was synthesized by reacting 10 g of 2-hydroxydiphenylmethane (Aldrich, 99%), 2 mmol oleic acid (Sigma-Aldrich, 90%) and 2 mmol Ni acetate (MCB, >99%) at 260° C. for 6 h followed by washing with ethanol and drying under vacuum for 10 h at 60° C., based on work of Leng et al. and Yang et al. [44, 45]. XRD (40 mA, 45 kV) of a coating scraped from a coupon employed a PANalytical Empyrean diffractometer with Cu Kα radiation. Spectra were recorded at 0.026° steps in the range 4°<20<70°. SEM images were obtained on a Quanta 3D FIB-SEM. The scraped coating was mounted directly onto carbon tape.

### 1.3. Reactor System Design.

The microreactor was fabricated by Mezzo Technology International, Baton Rouge, LA. Mezzo used its proprietary software to design the shell and tube sections of the microreactor system. The tubes are entirely of Inconel 625. The tubes contain the process gases while in the shell Dow Syltherm 800 flows as heat transfer fluid, countercurrently. Initially, heat transfer is in the direction of the tubes, but after the reaction exotherm the direction of heat transfer will reverse, from tubes to shell. The shell-side flow rate is controlled to attain the desired maximum and final process fluid temperatures. The tube core shape is annular. A picture of the tube core before it was inserted into the shell is shown below (FIG. 1). The microreactor tubes are laser welded to the tubesheets, which allows pressures >100 bar [46].

Key reactor design parameters are listed in Table 1:

TABLE 1
Microreactor Design/Operating Parameters

| Parameter | Value or Range |
|---|---|
| Maximum design flow, L/min | 3 at 70 bar |
| Methane/air ratio, molar | 2.1-3.0 |
| Outlet tube temperature, ° C. | 200-250 |
| Pressure drop, tubes, mbar | 0.2 |
| Inlet shell temperature, ° C. | 250 |
| Outlet shell temperature, ° C. | 250 |
| Pressure drop, shell, mbar | 0.2 |
| Tube core outer diameter, mm | 70 |
| Tube core inner diameter, mm | 10 |
| Heat transfer rate, calculated, shell to tubes, kW | 2.4 at 3.0 L/min |
| Maximum shell flow, L/min | 3 |
| Tube material | Inconel I625 |

The second section for quench is much shorter, 0.09 m, with a chilled copper tubing water jacket wrapped around this section. The jacket is controlled by a chiller. The microreactor is part of a high-pressure continuous flow system, with two electronic flow controllers, methane and air cylinders, a heating bath, an electrical resistance heating jacket, a backpressure regulator and a gas chromatograph (GC). This system is illustrated below in FIG. 2. There are separate temperature controls for the preheat section, the reactor core, the quench section, and the lines to the GC.

The current reactor consists of a single compartment where preheating, reaction and quenching all occur, but in alternative embodiments the system includes a second, separate quench compartment and a third preheat control compartment. On the basis of past work with simple tubular reactors, taking these steps would increase methanol yields further.⁵

The GC is an Agilent 5890, controlled by Chromperfect software. There are two columns/detectors, a ⅛″, 10 ft packed MS5A for the light gases, with a TCD detector, and a 0.53 mm, 30 m, 1.0 μm capillary AT-35 column for quantifying methane and methanol, by FID detector. For TCD calibration factors, we used standard tabulated ones, but checked these by injection of standards. FID calibration factors were determined by injections of standards.

### 1.4. Heat Transfer Control.

By moving a thermocouple up and down the length of the reactor outer shell wall, we determined that the reactor hot zone is only ˜8% of the total length, with a 3° C. measured variation. The hot zone was typically kept near 420-440° C., measured at the outer shell wall. In another embodiment a commercially distributed fiber optic sensor (e.g., of the type manufactured by Sensuron (https://www.sensuron.com/), will be installed in the 10 mm hole in the center of the tubesheet donut. Such a sensor allows rapid (>1 measurement/s) simultaneous temperature measurements along the entire length of the fiber, so the length of the reactor. This may allow better heat integration/control of the microreactor by allowing adjustment all the temperature controllers in real time to ensure optimal performance.

### 2. Results and Discussion

### 2.1. Carbiding Metal Coupons.

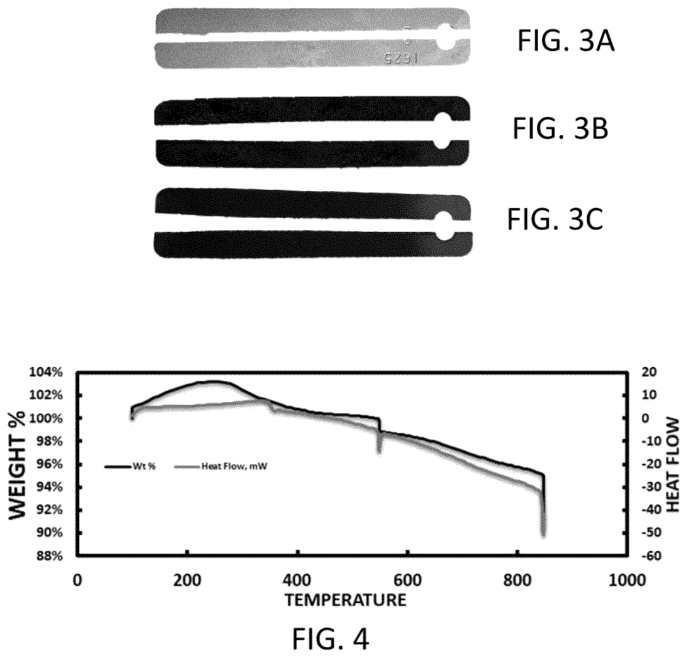

The carbide layers in this experiment were first deposited onto commercial corrosion coupons of Inconel I625, shown in FIG. 3a. The composition of I625 is roughly (wt %): Ni—60.6; Cr—22.2; Mo—8.3; Fe—4.4; Nb and Ta—3.4; Co—0.05; and minor amounts of Al, Ti, C, Mn, Si, P and S [48]. A coupon weighs 5.8-5.9 g, so from the weight gains given below it is seen that the coatings are thin.

Three methods of carbiding were tried, either using a graphite/inorganic salt mixture, or methane, or propane. In the graphite/salt method, the coupons were placed in a three-zone furnace (N₂, 300 mL/min) in an alundum combustion boat also containing the solid carbiding agent. The carbiding agent was graphite, 4 moles graphite per 1 mole of elemental Cr in the coupon. The graphite was mixed with inorganic salts that catalyze carbiding reactions, 58 wt % LiCl, 40 wt % KCl and 2% KF, with 1 g of salts per 0.3 g of Cr in the coupon (22.2 wt % Cr in a coupon). The salt mixture and graphite were ground using an agate mortar and pestle for 5 min. The temperature program for the furnace was 250 to 950° C. at 10° C./min. The weight gains for the two coupons were 87 mg (10 h hold at final temperature) and 52 mg (6 h hold) corresponding to calculated layer thicknesses of 55 and 33 μm. The coupons were shaken to remove excess material. Clearly this is a rapid synthesis, and an example coupon prepared by this method is shown in FIG. 3b.

In methane-based coating, the metal coupons were placed in a ½″ O.D. tubular reactor with α-alumina at the bottom to position the coupons in the hottest part of the reactor. Methane and nitrogen were fed to the reactor at flow rates of 20 and 35 cm³/min respectively, at 800° C. for six days. In both cases the coupon weight gains were 15 mg. In propane-based coating, N₂:propane at 2:1 molar ratio was the carrier gas using the same reactor as for methane. After 5 days at 700° C., 8-43 mg carbide could be formed. As this method is more adaptable to carbiding the entire microreactor without its removal from the system, it was the one adopted for subsequent work. An example coupon produced by this method is shown in FIG. 3c.

To test the reduction behavior of a coating, a scraped sample of a carbided coupon was reduced in 20% H₂/N₂ in the TGA/DSC (FIG. 4). The program was: 30 min, 100° C., 10° C./min to 350° C., 5° C./min to 550° C., 60 min at 550° C., 5° C./min to 850° C., 60 min at 850° C. At the lower temperatures, around 500-600° C., amorphous carbon and the more common iron carbides can be reduced [43]. At the higher temperatures, >700° C., graphite oxide can be reduced, but not the Cr carbides [38]. At 500° C., there is an ˜2% weight loss, at 850° C. ˜8%. This indicates that most of the layer is unreduced. The Cr carbides would also provide better oxidation resistance, reportedly up to 960° C. for the Cr carbides, far higher than the planned use temperatures.

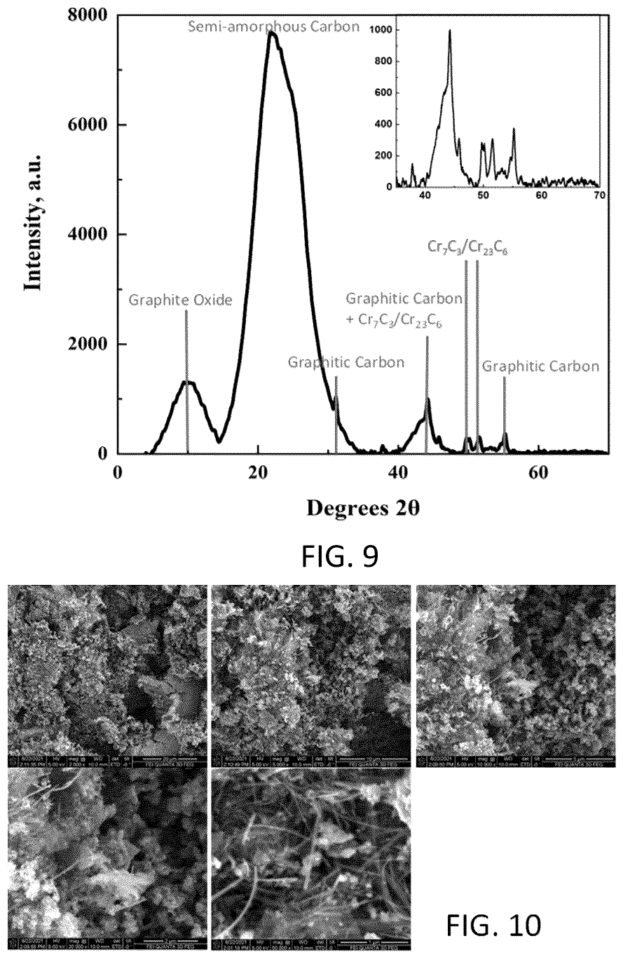

The XRD of a scraped layer from a carbided coupon (FIG. 9) shows clear evidence of graphite oxide (broad peak from 6-15° 2θ), [49, 50] semi-amorphous carbon (broad peak from from) 17-29°, [51-53] graphitic carbon (the shoulder peak at 26-27, 44.2, 55.2° 2θ) [54, 55] and the most common Cr carbide phases, Cr₇C₃ and Cr₂₃C₆ (peaks at 44.2, 49.7, 50.1, 51.5° 2θ) [38]. It is known that Cr carbide films deposited at 300° C. contain excess carbon, some graphitic, and in general are highly amorphous [56]. SEM images of this coupon (FIG. 10) show three regions, rough irregularly-shaped small particles (1, dominant structure), smooth-edged large crystals (2) and rods (3). Multiple regions of (1) and (2) were scanned by EDS. Structure (1) is almost entirely carbon. Structure (2) has composition (at %): C, 53±2; O, 35±2; Cr, 11±1; Ni, 0.2±0.1. This is broadly consistent with the XRD results, except no Cr₂O₃ was found by XRD.

We then tested the integrity of carbided coupons under oxidative conditions by mounting two coupons on a bed of circular MgAl₂O₄ monoliths in a 316 stainless steel cylindrical sample bomb. For O×3, one coupon was prepared by graphite-salt flux synthesis and one was prepared by the methane method. For O×4, both coupons were prepared by the propane method. The ceramic monoliths, themselves very inert, were further passivated by one week of carbide deposition from propane. Each integrity experiment lasted 24 h. Data are shown in Table 2. Because these needed to be accelerated tests, realistic temperatures and C1/air molar ratios, but lower pressures than anticipated in the reaction experiments were used. Lower pressures favor the desorption of the overlayers. The weights of the carbided coupons all either increased or only slightly decreased during these experiments. From this point onward we proceeded with a carbided (from propane decomposition) microreactor system.

TABLE 2
Results of Coupon Integrity Tests under Oxidizing Conditions

| Run # | Δm, % | T, ° C. | P, bar | C1/air | Coupon Type |
|---|---|---|---|---|---|
| Ox3 | 0.89 | 350 | 56.1 | 2.4 | Carbided via Salt |
| | 0.26 | | | | Carbided via Methane |
| Ox4 | −0.15 | 297 | 35.5 | 3.6 | Carbided via Propane |
| | 0.08 | | | | |

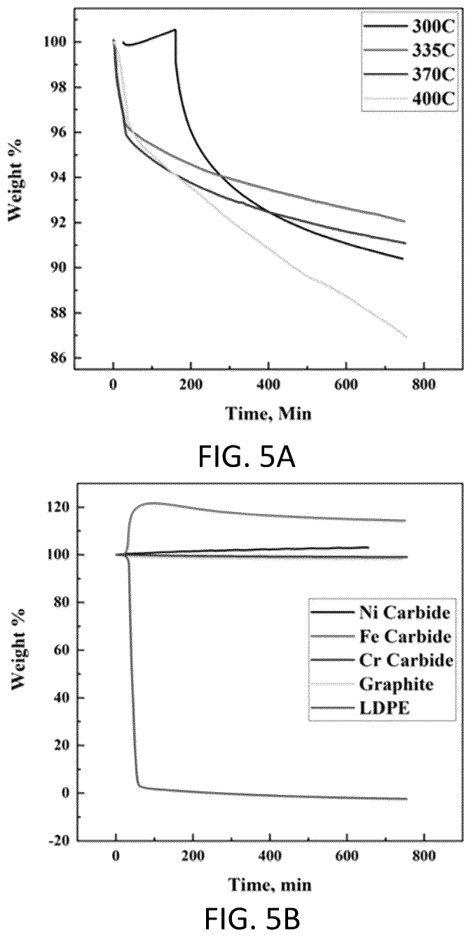

To further test the oxidation tolerance of the carbide/carbon layer, some scrapings were characterized in the TGA/DSC (FIG. 5a) at near atmospheric pressure. The carrier gas was 100 mL/min, C1/air=2.2. The program was: 10° C./min from 50° C. to the max temperature (330° C., 335° C., 370° C. or 400° C.) with a 12 h hold. It is evident that as the temperature increases, the layers are less stable but still relatively inert in that the weight derivatives are all decreasing at 12 h.

To identify which component of the carbided layers is least stable, a series of standards were also characterized in a similar manner, at 10° C./min from 50° C. to 400° C. with a 12 h hold. The graphite lost 2% of its weight, the Cr carbide 1%. The Ni carbide and Fe carbide gained weight, 3% and 14% respectively. A sample of low-density polyethylene (LDPE) lost 98% of its weight. The carbided sample weight losses are similar to that of graphitic carbon and not the much larger weight losses of the LDPE. Therefore, the weight losses observed in FIG. 5a can be identified primarily with the semi-amorphous graphite.

The average times it would take to lose 50% additional coating weight of the carbided samples are given below, in Table 3. These times were calculated from the weight loss derivatives for each run at the end of the 12 h hold. The numbers (which show no clear trend) also suggest that the weight changes in FIG. 5 are not solely due to carbon losses. There must be some oxidation of the carbides, which would increase weight, taking place as well. This is in agreement with the results in Table 2.

TABLE 3
Calculated Hours for 50% Additional Coating Weight Loss

| | 300° C. | 335° C. | 370° C. | 400° C. |
|---|---|---|---|---|
| Hours | 170 | 117 | 209 | 60.3 |

### 2.2. Carbiding the Microreactor Tubes.

The microreactor inner walls were carbided in a similar fashion as the coupons, using propane. The reaction to produce acetylenic carbon deposits can be written as:

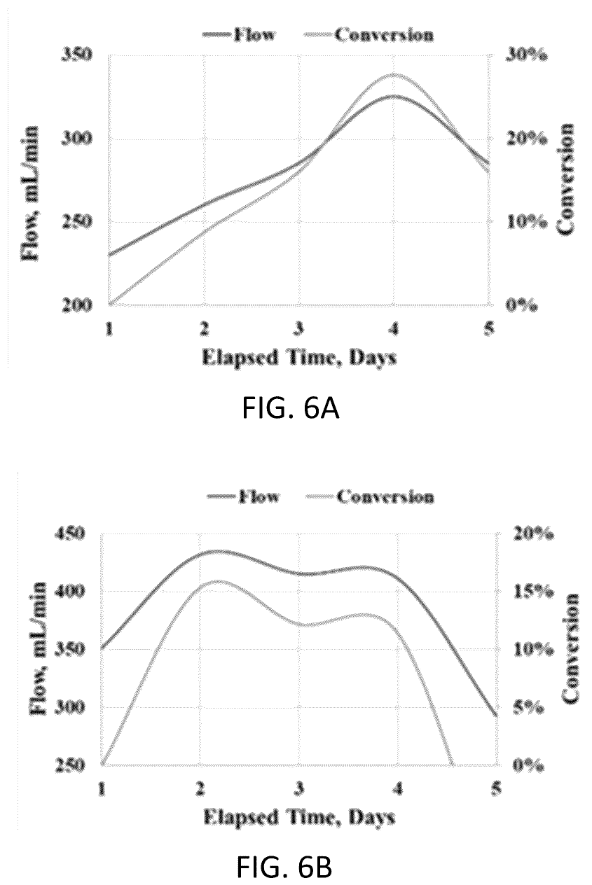

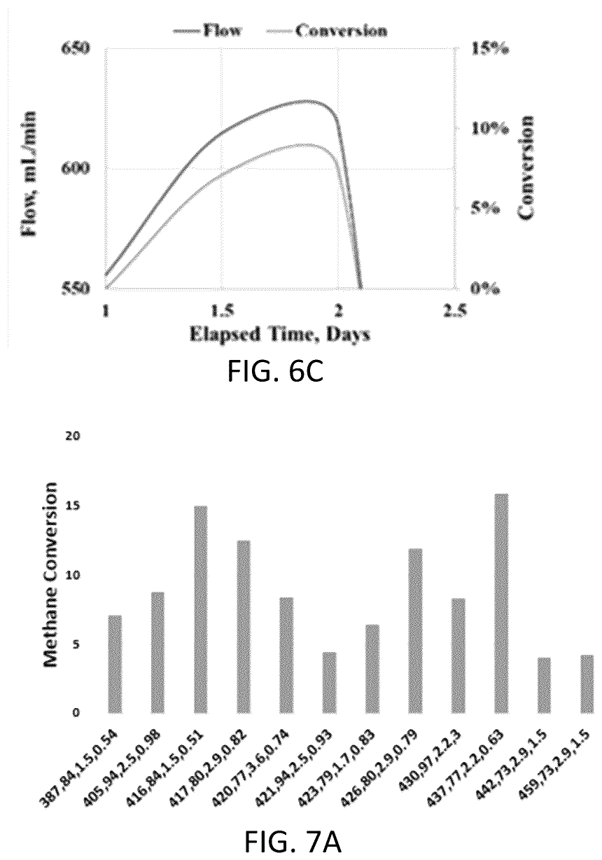

C₃H₈→3(CH)+2.5H₂

Where (CH) is a typical empirical formula for soft, non-graphitic, acetylenic coke. Because of the change in moles upon reaction, the total flow rate can be used to calculate the propane conversion. An increasing flow rate means an increase in conversion of the propane. But ultimately the flow rate decreases because the ΔP through the tubes was increasing significantly. This is when the propane flow and heating were discontinued. The results for three such tests are shown in FIG. 6a-c with the flow rates shown on day 1 the same as the feed flow rates. By integrating the conversions with respect to time, incremental layer thicknesses could be calculated: 137 μm for the first experiment, 116 μm for the second, and 30 μm for the third. The equations to perform these calculations are given in the Supplemental Information. However, the computations are by nature inexact because it is not known how evenly spread the carbided layer is. At lower temperatures, near the beginning and end of the reactor, there may have been little reaction of propane and little carbon deposition. The important result is that the decrease in the calculated incremental thicknesses suggests that partial carbide layers remained on the reactor tubes at the start of the second and third experiments. There were more DMTM reaction experiments between the first and second carbidings than between the second and third.

At the completion of each of the tests 1-3, the coated microreactor was used for the partial oxidation of a methane-air feed.

### 2.3. Methane Partial Oxidations in the Coated Microreactor.

The overall reactions at the conditions of this work are:

$$C {H}_{4} + \frac{1}{2} {O}_{2} \to C {H}_{3} OH$$

$$C {H}_{4} + 1.5 {O}_{2} \to C O + 2 {H}_{2} O$$

$$C {H}_{4} + 2 {O}_{2} \to {CO}_{2} + 2 {H}_{2} O$$

Small amounts of formaldehyde-methanol adducts were observed in a few runs but did not exceed 1% selectivity and are lumped with methanol in the data below (Table 4). All selectivities are on a mols carbon basis, and details of the algorithm are in the Supporting Information. The errors in C1 conversion and CH₃OH selectivity were computed from the precisions of the flow rates and GC calibration factors. Each entry in the table is an average for a particular day or half-day.

TABLE 4
Results of DMTM Experiments that Produced Methanol, Carbided Microreactor¹

| | Reactor | | | | | | | | |
|---|---|---|---|---|---|---|---|---|---|
| Entry | Wall T, | P, | C1/Air, | RT, | % C | | MeOH | CO | CO₂ |
| Number | ° C. | bar | molar | min | balance | C1 Conv. | % Sel | % Sel | % Sel |
| 1 | 387 | 84 | 1.5 | 0.54 | 93 | 7.1 ± 0.3 | 69 ± 16 | 31 | 0 |
| 2 | 416 | 84 | 1.5 | 0.51 | 97 | 15 ± 0.7 | 14 ± 3 | 49 | 37 |
| 3 | 423 | 79 | 1.7 | 0.83 | 103 | 6.4 ± 0.3 | 6 ± 1 | 6 | 88 |
| 4 | 437 | 77 | 2.2 | 0.63 | 99 | 15.9 ± 0.7 | 62 ± 15 | 24 | 14 |
| 5 | 430 | 97 | 2.2 | 3.0 | 99 | 8.3 ± 0.4 | 0.0 | 68 | 32 |
| 6 | 405 | 94 | 2.5 | 0.98 | 100 | 8.8 ± 0.4 | 54 ± 13 | 39 | 7 |
| 7 | 421 | 94 | 2.5 | 0.93 | 104 | 4.4 ± 0.2 | 20 ± 5 | 45 | 35 |
| 8 | 442 | 73 | 2.9 | 1.5 | 100 | 4.0 ± 0.2 | 34 ± 8 | 66 | 0 |
| 9 | 459 | 73 | 2.9 | 1.5 | 100 | 4.2 ± 0.2 | 5 ± 1 | 62 | 33 |
| 10 | 417 | 80 | 2.9 | 0.82 | 98 | 12.5 ± 0.6 | 80 ± 19 | 20 | 0 |
| 11 | 426 | 80 | 2.9 | 0.79 | 97 | 11.9 ± 0.6 | 81 ± 19 | 19 | 0 |
| 12 | 420 | 77 | 3.6 | 0.74 | 94 | 8.4 ± 0.4 | 67 ± 16 | 15 | 18 |

¹MeOH = methanol, Conv = conversion, Sel = selectivity. All selectivities are on a mols carbon basis. All quantities are averages of multiple GC runs.

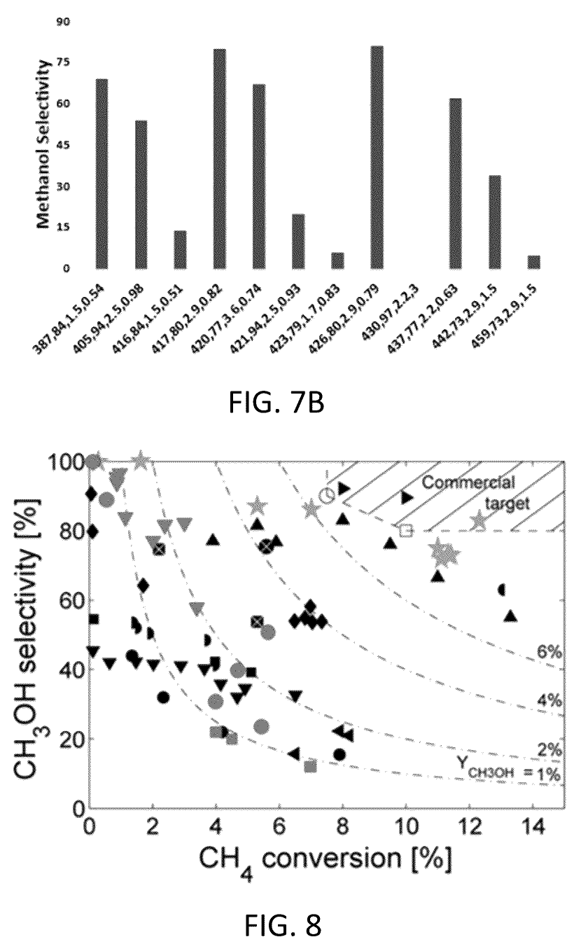

The initial trials (not shown in Table 4 or FIG. 7) were with no coating. There was little to no oxidation of methane at 2.2:1 C1/air molar ratio (320 mL/min total flow at NTP, 73-77 bar) at up to 380° C. on the reactor wall. At higher temperature methane was oxidized but mostly to CO and CO₂.

The methanol left the hot reaction zone and then had to be quenched by a rapid temperature drop. We found that this final quench temperature must be less than ˜250° C. to stop the further reaction of methanol, but greater than 150° C. to prevent condensation of water in the lines. Experiments where the final temperatures were >250° C. gave poor selectivity to methanol. Experiments with final temperatures <150° C. gave poor carbon balances. These results are also not presented in Table 4.

Later work was done with coated walls via the propane decomposition method described above, and this work established that the optimal wall temperature of the coated microreactor is ˜380-440° C. with a final quench temperature ˜200-250° C. However, the data in Table 4 taken over these temperature ranges do not show a consistent increase or decrease in methanol selectivity with respect to just reactor wall temperature. For example, compare runs 2 and 10, or 3 and 11, those with similar wall temperatures. Other factors, such as the C1/air molar ratio, residence time and pressure play a significant role. It is true according to these results that it is not possible to have a high methanol selectivity at C1/air <2.2, unless the reactor temperature is <400° C., the pressure relatively high, and the residence time short (first row in Table 4). At very long residence times (e.g., entries 5, 8 and 9 of Table 4), the methanol is always over-oxidized.

A key result is that per pass methanol yields in excess of 8% (entries 4, 10 & 11) and selectivities >70% (entries 10 & 11) are possible given a high enough (2.9) C1/air ratio and pressure. High yields are still possible at ratios of 2.5 or less, if other conditions are favorable. These results are significant since the studies discussed previously indicate an economic tipping point of >8% yield at >70% selectivity [2]. However, there was evidence that as the runs get further in time from the initial reactor carbiding, the selectivity to methanol was decreasing. For example, the time elapsed post carbiding for the last run in the Table was more than twice that of the preceding two runs—the loss of selectivity is obvious in FIG. 7b. In general, experiments run more than two days after the carbiding reactions produced less methanol. This also suggests, in agreement with FIG. 5a, that the carbide layer is being gradually stripped off and/or oxidized, and must eventually be replenished.

It would be expected that a stripped carbide layer would result in some aromatic products. Liquid samples collected after three different runs, obtained by condensing the effluent, showed small amounts of ethanol, toluene, propanol, butanol, and acetic acid by GC/MS. The higher alcohols and acetic acid are expected given the methanol production, but not so the toluene. There were also small amounts of black particulates in this condensate which were carbon. Therefore the carbide layers are not fully stable and must be made more so in the future-just as all the catalysts now used for the air-oxidized methane to methanol reaction are unstable, more so than here.

### 2.4. Discussion.

The reason the microreactor provides more precise temperature control is because microreactors in general transfer more heat transfer per volume than larger diameter tubes. The UA/volume scales as 1/D2 [46]. For 1 mm diameter tubes the heat transfer rate is 150 times greater than for more conventional ½″ tubes.

FIG. 8 shows compiled experimental results for DMTM to 2008, redrawn and updated to bring the results up through 2015, for both homogeneous (black symbols) and catalytic (red symbols) DMTM [3, 5, 31, 35, 57-66]. In the 80-90's, several groups using homogeneous DMTM processes, including Gesser et al. (the upward-[3] and right-pointing [57] black solid triangles) and this group (blue stars) [5] reached what has been identified as a desired commercial target, shown in the top right corner [2, 28]. Another group has come reasonably close (left-half circle) [7] to reproducing the older results. The current results show that we are in or near the commercial target range, but without reliance on exotic/fragile wall materials, hot-wire filament or low-temperature plasma initiation, or chemical sensitizers, all of which would contribute greatly to the cost of methanol production.

In contrast to the homogeneous results, the best reported heterogeneous catalysts for DMTM with O₂ as oxidant (at up to ˜650° C.) give only 1-5% yields, predominantly to less valuable formaldehyde [60]. The exceptions are processes using soluble catalysts in aqueous acids and expensive oxidants such as H₂O_{2 [67}] or periodate [68], both at low oxidant efficiency. All methane conversion processes involve breaking the stable C—H bond, initially forming a methyl radical (•CH₃). Catalysts can activate this step, but at normal conversions cannot stop here. The catalyst abstracts additional hydrogens, thus promoting deep oxidation to CO₂ [33, 34, 58-61, 69, 70]. Our strategy for successful DMTM does not focus on catalyst development, but rather the understanding that high yields for DMTM require better control of certain gas-phase chain branching reactions characteristic of the so-called “cool flame” region, [28, 34, 71] through better temperature control, and the elimination of O₂ and methane chemisorption on solid surfaces.

In the past few years, the catalytic methane to methanol process has undergone extensive reworking by operating at higher pressures or in the presence of H₂O₂ as oxidant. At high pressure, there is no way to know how much of the methanol is produced catalytically on a surface, how much is produced by homogeneous reactions, or if the two processes are interacting. But the processes can be compared roughly, on a “productivity” basis, g MeOH/(g cat·h) produced. Since ours is a non-catalytic process there is no way to make a direct comparison, but we made one by using the weight of the tubes of the microreactor instead of catalyst weight. In fact, the other processes would need multitube reactors to work also, so this is highly conservative in favor of the other processes. The data for the other processes also do not show how long the catalyst could last.

From our own work, the highest productivity using the data in Table 2 is 3.0×10⁻² g/(g reactor·h). For a typical semi-continuous catalytic process, the best published productivity is for a Cu (9.32 wt %) faujasite catalyst, Si/Al=2.6 [72]. They obtained 360 μmol methanol/gcat, which corresponds to a productivity of 4.1×10⁻³ g methanol/(gcat·h). The methanol was obtained at 50 mL/min methane, total pressure 8 bar for 30 min, followed by He purge for 20 min, then methanol desorption in 2.6 vol % H₂O/He at 473 K, 1 bar for 1 h. If using H₂O₂ as oxidant, a representative study is that of Yu et al. [73]. Their best catalyst/reactor conditions were 2% (wt) Cu, 0.1% Fe/MFI zeolite, for partial oxidation in a batch reactor at 50° C., 30 bar methane, 0.5 M H₂O₂. They obtained 431 mol MeOH/(mol Fe·h) at 80% selectivity. This corresponds to 0.25 g MeOH/(gcat·h), but it must be noted that catalyst stability was not evaluated, and H₂O₂ is a far more expensive (but effective) oxidant than air.

Our preliminary conclusions are that the carbide layer will be partly stripped off in ˜5 runs at typical conditions, meaning roughly 25 h of run time. The loss of part of the carbide layer negatively affects the performance of the reactor, but the decay in conversion/selectivity cannot be quantified from the present data set. We are attempting to stabilize the coating by converting it from a pure carbide to a mixed carbide-nitride, carbide-boride, or carbide-sulfide. The chemistry to make such coatings on high Ni, Cr alloys is known and coating conditions are accessible with our current apparatus. We will initially test I625 coupons to determine the optimal coating times and conditions (temperature, reagent partial pressures or liquid rates, and coating times). Once these are determined, we will decide upon one of the alternative methods (carbide-nitride, carbide-boride, carbide-sulfide), coat the microreactor by this method, and then run further reaction tests to determine both reactor performance and coating stability. For example, we have found that TEPA (tetraethylenepentamine) reacted with carbided Inconel 625 gives a highly nitrided carbon coating at accessible temperatures (carbiding 750° C., 4 d, nitriding 400° C., 6 d). We expect this coating to be more resistant to oxidation on the microreactor walls during methane to methanol high-pressure partial oxidation.

### 3. Conclusions

Methanol was selectively produced with per pass yields>8% and selectivities >70% by controlling the five key parameters (temperature, pressure, residence time, wall inertness and methane:air ratio) under optimal conditions in an integrated mixer-reactor-heat exchanger microtube scalable system. The optimal conditions are a total pressure near 80 bar, 420° C. outer wall temperature, a C1/Air molar ratio ˜2.9 and a residence time of ˜0.8 min. At lower C1/air molar ratios (2.5 and less) high yields were still possible, but at the price of lower methanol selectivity. The results are competitive with the commercial two-step, catalytic process. Importantly, methanol was selectively made without the use of exotic wall materials, hot-wire filament or plasma radical initiation, sensitizers (chemical radical initiators), sophisticated mixing designs, or any combination of these.

The carbiding of the microreactor walls was by the decomposition of propane at near 700° C. However, the carbide layers are not perfectly stable and some carbon and carbide oxidation was observed in TGA experiments. These transformations affected the selectivities to methanol the further removed in time from reactor carbiding.

### Example 1, Appendix A. Supplementary Data

### Reactions and Extents of Reactions

ξ₁:CH₄+2O₂→CO₂+2H₂O

ξ₂:CH₄+1.5O₂→CO+2H₂O

ξ₃:CH₄+0.5O₂→CH₃OH

There are seven mass balances, where Fₜ=total outlet flowrate, F₁-F₅ and Fₐᵢᵣ are the feed flowrates, but in most cases F₂-F₅ are zero. These flow rates are in mol/min. The ξ's are also in mol/min. The Y's are the corrected (true) product mole fractions.

$${CH}_{4} : {F}_{t} * {Y}_{1} = {F}_{1} - {ξ}_{1} - {ξ}_{2} - {ξ}_{3}$$

$${CO}_{2} : {F}_{t} * {Y}_{2} = {F}_{2} + {ξ}_{1}$$

$${H}_{2} O : {F}_{t} * {Y}_{3} = {F}_{3} + 2 {ξ}_{1} + 2 {ξ}_{2}$$

$$CO : {F}_{t} * {Y}_{4} = {F}_{4} + {ξ}_{2}$$

$${CH}_{3} OH : {F}_{t} * {Y}_{5} = {F}_{5} + {ξ}_{3}$$

$${O}_{2} : {F}_{t} * {Y}_{6} = {F}_{a i r} * 0.21 - 2 {ξ}_{1} - 1.5 {ξ}_{2} - 0.5 {ξ}_{3}$$

$${N}_{2} : {F}_{t} * {Y}_{7} = {F}_{a i r} * 0.79$$

The Y's are determined using N₂ as an internal standard in the TCD analysis. The y's are the uncorrected mole fractions from the TCD analysis (which cannot account for methanol), and the x's are the uncorrected mole fractions from the FID analysis (which cannot account for N₂, O₂, CO, CO₂ or H₂O). The Y's are solved for from the following equations, then normalized.

$${N}_{2} : {Y}_{7} = ( {F}_{a i r} * 0.79 ) / {F}_{t}$$

$${CH}_{4} : {Y}_{1} = {Y}_{7} * ( {y}_{1} / {y}_{7} )$$

$${CO}_{2} : {Y}_{2} = {Y}_{7} * ( {y}_{2} / {y}_{7} )$$

$$CO : {Y}_{4} = {Y}_{7} * ( {y}_{4} / {y}_{7} )$$

$$C {H}_{3} OH : {Y}_{5} = {Y}_{1} * ( {x}_{5} / {x}_{1} )$$

$${O}_{2} : {Y}_{7} * ( {y}_{6} / {y}_{7} )$$

$${H}_{2} O : {Y}_{3} = 2 * ( {Y}_{2} + {Y}_{4} )$$

Two solutions of the above equations were compared in solving for the ξ's. The first (“Nonlinear Regression”) assumes Fₜ is not known, while the second (“Direct Solution”) uses Fₜ in the solution.

### 1. Nonlinear Regression.

Calculate Fₜ=Σ₁⁵ Fᵢ+Fₐᵢᵣ+0.5*(ξ₂−ξ₃) to use in the mass balances. Subtract the right-hand side from the left-hand side of each mass balance, and solve for the ξ's using the objective function:

$$O . F . = {( L H {S}_{1} - R H {S}_{1} )}^{2} + {( L H {S}_{2} - R H {S}_{2} )}^{2} + {( L H {S}_{4} - R H {S}_{4} )}^{2} + {( LH {S}_{5} - R H {S}_{5} )}^{2} + {( L H {S}_{6} - R H {S}_{6} )}^{2} + {( L H {S}_{{F}_{t}} - R H {S}_{{F}_{t}} )}^{2}$$

The extents ξ₁, ξ₂ and ξ₃ are solved for by minimizing this objective function using the ξ's from the direct solution as the initial guesses.

### 2. Direct Solution-Assumes Fₜ is Known

Fₜ=Fₒᵤₜ/(1−Y₃−Y₅); Fₒᵤₜ is the measured outlet gas flow, after condensing the water and methanol.
Solve for ξ₁, ξ₂, and ξ₃ using the mass balances and computed total flow, Fₜ

$${ξ}_{1} = {F}_{t} * {Y}_{2} - {F}_{2}$$

$${ξ}_{2} = {F}_{t} * {Y}_{4} - {F}_{4}$$

$${ξ}_{3} = {F}_{t} * {Y}_{5} - {F}_{5}$$

### Carbon Balance, Conversions, Yields and Selectivities

Carbon balance, mole basis: 1−(F₁−Fₜ*(Y₁+Y₂+Y₄+Y₅))/F₁
CH₄ conversion=(ξ₁+ξ₂+ξ₃)/F₁
O₂ conversion: (Fₐᵢᵣ*0.21−Fₜ*Y₆)/(Fₐᵢᵣ*0.21)
Theoretical O₂ conversion=(2*ξ₁+ξ₂+0.5ξ₃)/(0.21*Fₐᵢᵣ)
CH₃OH yield=ξ₃/F₁
CO yield=ξ₂/F₁
CH₃OH selectivity=ξ₃/(ξ₁+ξ₂+ξ₃)
CO selectivity=ξ₂/(ξ₁+ξ₂+ξ₃)
Residence Time in the hot zone: ((π*0.25*D²*L_{tube}*N_{tubes}*(frac of L in hot zone))/((V₁+Vₐᵢᵣ)*(1.013/P_{system})*(T_{wall}+273.2)/(Tₐₜₘ+273.2)) V₁ and Vₐᵢᵣ are the volumetric flow rates of CH₄ and air entering the system.

### Layer Thickness Calculations

### Microtubes:

$$X = \frac{( \frac{{F}_{out}}{{F}_{i n}} - 1 )}{1.5}$$

$$T = \frac{3 ( \int X dt ) ( {F}_{i n} {ρ}_{M} M {W}_{c} )}{{S}_{tot} {ρ}_{c}}$$

Where X is the fraction conversion of propane, Fₒᵤₜ and Fᵢₙ are the outlet and inlet volumetric flow rates, ρ_{M} is the molar density of propane, MW_{c} the molecular weight of the carbon (13), Sₜₒₜ the inner surface area of all the tubes, ρ_{c} the mass density of carbon, and the “3” accounts for three mols of carbon produced from one mol propane.

$$Coupon := ( mass increase , g ) / [ ( density of C , g / {cm}^{3} ) * [ 2 {( width * length + height * length + height * width )}_{coupon} - ( π * {D}_{hole i n coupon}^{2} * 2 ) + ( π * {D}_{hole i n coupon}^{2} * 0.1 ) , {cm}^{2} ]$$

### Equations for GC Calibrations

$$GC Absolute Factor = ( moles injected ) / ( area of peak )$$

$$GC relative Factor = ( component factor ) / ( base factor ) ,$$

$$i . e . ( REL ) j / ( REL ) i = ( ABS ) j / ( ABS ) i$$

For the FID analysis, CH₄ is the base factor.
For the TCD analysis, N₂ is the base factor
% Error in GC factors: (standard deviation of data)/(averaged value of data)

### REFERENCES FOR EXAMPLE 1

[1] P. Khirsariya, Single Step Oxidation of Methane to Methanol-Towards Better Understanding, Procedia Engineering 51 (2013) 409-415. https://doi.org/10.1016/i.proeng.2013.01.057.
[2] E. M. Turan, E. van Steen, K. P. Möller, Comparison of mechanisms for the direct, gas phase, partial oxidation of methane to methanol Chem. Eng. Sci. 241 (2021) 116718.
[3] P. S. Yarlagadda, L. A. Morton, N. R. Hunter, H. D. Gesser, Direct conversion of methane to methanol in a flow reactor, Industrial and Engineering Chemistry Research 27 (2) (1988) 252-256.
[4] N. R. G. Hunter, H. D.; Morton, L. A.; Yarlagadda, P. S.; Fung, D. P. C., Methanol formation at high pressure by the catalyzed oxidation of natural gas and by the sensitized oxidation of methane, Applied Catalysis 57 (1990) 815-822.
[5] W. Feng, F. C. Knopf, K. M. Dooley, Effects of pressure, third bodies, and temperature profiling on the noncatalytic partial oxidation of methane, Energy & Fuels 8 (4) (1994) 815-822.
[6] Q. J. Zhang, D. H. He, X. Zhang, Q. M. Zhu, S. L. Yao, Direct partial oxidation of methane to methanol in a specially designed reactor, in: C. J. Liu, R. G. Mallinson, M. Aresta (Eds.), Utilization of Greenhouse Gases, ACS, Washington, D.C., 2003, pp. 280-290.
[7] Q. J. Zhang, D. H. He, J. L. Li, B. Q. Xu, Y. Liang, Q. M. Zhu, Comparatively high yield methanol production from gas phase partial oxidation of methane, Applied Catalysis A: General 224 (1-2) (2002) 201-207. https://doi.org/10.1016/s0926-860x(01)00820-1.
[8] T. Nozaki, A. Ağiral, S. Yuzawa, J. G. E. Han Gardeniers, K. Okazaki, A single step methane conversion into synthetic fuels using microplasma reactor, Chemical Engineering Journal 166 (1) (2011) 288-293. https://doi.org/http://dx.doi.org/10.1016/i.cei.2010.08.001.
[9] T. Nozaki, K. Okazaki, Non-thermal plasma catalysis of methane: Principles, energy efficiency, and applications., Catal. Today 211 211 (2013) 29-38.
[10] H. Puliyalil, D. L. Jurkovic, V. D. B. C. Dasireddy, B. Likozar, A review of plasma-assisted catalytic conversion of gaseous carbon dioxide and methane into value added platform chemicals and fuels, RSC Adv. 8 (2018) 27481-27508.
[11] Y. Yi, S. Li, Z. Cui, Y. Hao, Y. Zhang, L. W. Wang, P. Liu, X. Tu, X. Xu, H. Guo, A. Bogaerts, Selective oxidation of CH₄ to CH₃OH through plasma catalysis: Insights from catalyst characterization and chemical kinetics modelling, Appl. Catal. B: Environ. 296 (2021) 120384.
[12] P. Chawdhury, Y. Wang, D. Ray, S. Mathieu, N. Wang, J. Harding, F. Bin, X. Tu, C. Subrahmanyam, A promising plasma-catalytic approach towards single-step methane conversion to oxygenates at room temperature, Appl. Catal. B: Environ. 284 (2021) 119735.
[13] K. Bullian, Optimizing Methanol Usage for Hydrate Inhibition in a Gas Gathering System 83rd Annual GPA Convention (2004).
[14] J. Beath, N. Black, M. Boone, G. Roberts, B. Rutledge, A. Elgowainy, M. Wang, J. Kelly, Contribution of Infrastructure to Oil and Gas Production and Processing Carbon Footprint, U.S. Energy Information Administration, Washington, D.C., 2014.
[15] R. Fullenbaum, J. Fallon, B. Flanagan, Oil & Natural Gas Transportation & Storage Infrastructure: Status, Trends, & Economic Benefits, American Petroleum Institute, Washington, D.C., 2013.
[16] EIA, Natural Gas Processing: The Crucial Link Between Natural Gas Production and Its Transportation to Market, U.S. Energy Information Administration, Washington, D.C., 2006.
[17] EIA, Trends in U.S. Oil and Natural Gas Upstream Costs, U.S. Energy Information Administration, Washington, D.C., 2016.
[18] Anadarko Petroleum Corporation and the Domestic Petroleum Council, Natural Gas Dehydration: Lessons Learned from the Natural Gas STAR Program, U.S. EPA, Washington, D.C., 2007.
[19] S. Mokhatab, W. A. Poe, J. Y. Mak, Handbook of Natural Gas Transmission and Processing: Principles and Practices, Elsevier, Oxford, U K, 2015.
[20] C. Cooley, B. K. Wallace, R. Gudimetla, Hydrate Prevention and Methanol Production on Canyon Express, SPE Annual Technical Conference and Exhibition, Society of Petroleum Engineers, Denver, 2003.
[21] ProSim, Natural Gas Dehydration Unit with Triethylene Glycol, ProSim Corp., Labege, France, 2010.
[22] SPE, Predicting hydrate formation, last accessed 5/17/17. http://petrowki.org/Predicting_hydrate_formation. (Accessed 5/17/17.
[23] Methanol Institute—Wastewater Treatment, 2017. http://www.methanol.org/. (Accessed 5/17/17.
[24] CDM Inc.—Evaluation of methanol feed, storage and handling costs at municipal wastewater treatment facilities, 2007.
[25] K. Law, J. Rosenfeld, M. Jackson, Methanol as a Renewable Energy Resource, The Methanol Institute, Alexandria, V A, 2013.
[26] E. J. Moniz, H. D. Jacoby, A. J. M. Meggs, R. C. Armstrong, D. R. Cohn, S. R. Connors, J. M. Deutch, Q. J. Ejaz, J. S. Hezir, G. M. Kaufman, M. A. Kenderdine, F. O'Sullivan, S. Paltsev, J. E. Parsons, I. Perez-Arriaga, J. M. Reilly, C. Seto, M. D. Webster, Y. Yang, G. S. McRae, C. Ruppel, The Future of Natural Gas: An Interdisciplinary MIT Study, MIT, Cambridge, MA, 2011.
[27] R. M. Noyes, Effects of diffusion rates on chemical kinetics, Progr. in Reaction Kinetics (G. Porter, editor. Pergamon Press) 1 (1961) 129-160.
[28] V. Arutyunov, Direct Methane to Methanol: Foundations and Prospects of the Process, Elsevier, Oxford, U K, 2014.
[29] C. L. Rasmussen, J. G. Jakobsen, P. Glarborg, Experimental Measurements and Kinetic Modeling of CH4/O₂ and CH₄/C₂H₈/O₂ Conversion at High Pressure, International Journal of Chemical Kinetics 40(12) (2008) 778-807. https://doi.org/10.1002/kin.20352.
[30] C. L. Rasmussen, P. Glarborg, Direct partial oxidation of natural gas to liquid chemicals: Chemical kinetic modeling and global optimization, Industrial & Engineering Chemistry Research 47(17) (2008) 6579-6588. https.//doi.org/10.1021/ie800137d.
[31] Q. Zhang, D. He, X. Zhang, Q. Zhu, S. Yao, Direct Partial Oxidation of Methane to Methanol in a Specially Designed Reactor, in: C. J. Liu, R. G. Mallinson, M. Aresta (Eds.), Utilization of Greenhouse Gases, ACS, Washington, D C, 2009, pp. 280-290.
[32] V. S. Arutyunov, The role of pressure in partial oxidation of methane, Russian Chemical Bulletin 51(12) (2002) 2170-2175. https://doi.org/10.1023/A:1022114830675.
[33] V. S. Arutyunov, V. M. Rudakov, V. I. Savchenko, E. V. Sheverdenkin, O. G. Sheverdenkina, A. Y. Zheltyakov, Partial alkane oxidation kinetics at high pressure: methane oxidation in stainless steel and quartz reactors, Theoretical Foundations of Chemical Engineering 36(5) (2002) 472-476.
[34] C. K. Westbrook, Y. Mizobuchi, P. J. Poinsot, P. J. Smith, J. Warnatz, Computational combustion, Proc. Comb. Inst. 30 (2005) 125-157.
[35] R. Burch, G. D. Squire, S. C. Tsang, Direct conversion of methane into methanol, J. Chem. Soc., Faraday Trans. 1 85 (1989) 3561.
[36] V. Arutyunov, Partial Alkane Oxidation Kinetics at High Pressures: Methane Oxidation in Stainless Steel and Quartz Reactors, Theoretical Foundations of Chemical Engineering 36 (2002) 472-476. https://doi.org/10.1023/A:1020677829300
[37] T. Kim, Chromium Carbide Laser-Beam Surface-Alloying Treatment on Stainless Steel, Journal Mater. Sci. 27 (1992) 2967-2973.
[38] S. M. Schmuecker, D. Clouser, T. J. Kraus, B. M. Leonard, Synthesis of Metastable Chromium Carbide Nanomaterials and Their Electrocatalytic Activity for the Hydrogen Evolution Reaction, Dalton Trans. 46 (2017) 13524-13530.
[39] N. Srisuwan, The Study of Heat Treatment Effects on Chromium Carbide Precipitation of 35Cr-45Ni—Nb Alloy for Repairing Furnace Tubes, Metals 6 (2016). https://doi.org/10.3390/met6010026.
[40] K. Law, Methanol as a Renewable Energy Resource, The Methanol Institute, Alexandria, VA2013.
[41] S. Schmuecker, Synthesis of Metastable Chromium Carbide Nanomaterials and Their Electrocatalytic Activity for the Hydrogen Evolution Reaction, Dalton Trans 46 (2017) 13524-13530. https://doi.org/10.1039/c7dt01404j.
[42] V. Arutyunov, Direct Methane To Methanol: Foundations and Prospects of the Process., Direct Methane to Methanol (2014) vii-ix. https://doi.org/10.1016/b978-0-444-63253-1.02001-8.
[43] T. Herranz, S. Rojas, F. J. Perez-Alonso, M. Ojeda, P. Terreros, J. L. G. Fierro, Genesis of iron carbides and their role in the synthesis of hydrocarbons from synthesis gas, J. Catal. 243 (2006) 199-211.
[44] Y. L. H. S. Y. W. M. S. a. X. Li, Nickel Carbide (Ni3C) Nanoparticles in Solution, Journal of Nanoscience and Nanotechnology 6 (2006) 221-226.
[45] J. Yang, X. Zhang, X. Zhou, Y. Hong, J. Shao, Y. Zhang, Q. Yan, X. Dong, Controlled synthesis of nickel carbide nanoparticles and their application in lithium storage, Chemical Engineering Journal 352 (2018) 940-946. https://doi.org/10.1016/j.cej2018.06.266.
[46] M. Technology, Why Microtubes? http://mezzotech.com/why-microtubes.html.
[47] W. A. Dietz, Response Factors for Gas Chromatographic Analyses, J. Gas Chrom. 5 (1967) 68-71.
[48] E. S. M. Corp, Inconel Alloy 625, 2013. specialmetals.com/assets/smc/documents/alloys/inconel/inconel-alloy-625.pdf.
[49] S. Tourani, A. M. Rashidi, A. A. Safekordi, H. R. Aghabozorg, F. Khorasheh, Synthesis of Reduced Graphene Oxide-Carbon Nanotubes (rGO-CNT) Composite and Its Use As a Novel Catalyst Support for Hydro-Purification of Crude Terephthalic Acid, Ind. Eng. Chem. Res. 54 (2015) 7591-7603.
[50] H. Cheun Lee, W. W. Liu, S. P. Chai, A. R. Mohamed, A. Aziz, C. S. Khe, N. M. S. Hidayah, U. Hashim, Review of the synthesis, transfer, characterization and growth mechanisms of single and multilayer graphene, RSC Adv. 7 (2017) 15644.
[51] Z. Yang, R. Mokaya, Probing the effect of the carbonisation process on the textural properties and morphology of mesoporous carbons, Micro. Meso. Mater. 113 (2008) 378-384.
[52] L. Liu, F. Y. Wang, G. S. Shao, Z. Y. Yuan, A low-temperature autoclaving route to synthesize monolithic carbon materials with an ordered mesostructure, Carbon 48 (2010) 2089-2099.
[53] M. Noh, Y. Kwon, H. Lee, J. Cho, Y. Kim, M. G. Kim, Amorphous Carbon-Coated Tin Anode Material for Lithium Secondary Battery, Chem. Mater. 17 (2005) 1926-1929.
[54] PDF #00-056-0159, International Centre for Diffraction Data, Cambridge, UK, 2013.
[55] PDF #03-065-6212, International Centre for Diffraction Data, Cambridge, UK, 2013.
[56] M. Magnuson, M. Andersson, J. Lu, L. Hultman, U. Jansson, Electronic Structure and Chemical Bonding of Amorphous Chromium Carbide Thin Films, J. Phys. Cond. Matter 24 (2012) 225004.
[57] N. R. Hunter, H. D. Gesser, L. A. Morton, P. S. Yarlagadda, D. P. C. Fung, Methanol formation at high pressure by the catalyzed oxidation of natural gas and by the sensitized oxidation of methane, Applied Catalysis 57 (1) (1990) 45-54. https://doi.org/10.1016/S0166-9834 (00) 80722-8.
[58] Q. J. Zhang, D. H. He, Z. S. Han, H. Zhang, Q. M. Zhu, Controlled partial oxidation of methane to methanol/formaldehyde over Mo—V—Cr—Bi—Si oxide catalysts, Fuel 81 (11-12) (2002) 1599-1603. https://doi.org/10.1016/s0016-2361 (02) 00076-5.
[59] X. W. Wang, Y.; Tang, Q.; Guo, Q.; Zhang, Q.; Wan, H., MCM-41-supported iron phosphate catalyst for partial oxidation of methane to oxygenates with oxygen and nitrous oxide, J. Catal. 217 (2003) 457-467.
[60] H. Berndt, A. Martin, A. Bruckner, E. Schreier, D. Muller, H. Kosslick, G. U. Wolf, B. Lucke, Structure and Catalytic Properties of VOX/MCM Materials for the Partial Oxidation of Methane to Formaldehyde, J. Catal. 191 (2000) 384-400.
[61] S. Pei, B. Yue, L. Qian, S. Yan, J. Cheng, Y. Zhou, S. Xie, H. He, Preparation and characterization of P—Mo—V mixed-oxide incorporating mesoporous silica catalysts for selective oxidation of methane to formaldehyde, Appl. Catal. A: Gen. 329 (2007) 148-155.
[62] I. Bar-Nahum, A. M. Khenkin, R. Neumann, Mild, Aqueous, Aerobic, Catalytic Oxidation of Methane to Methanol and Acetaldehyde Catalyzed by a Supported Bipyrimidinylplatinum-Polyoxometalate Hybrid Compound, J. Am. Chem. Soc. 126 (2004) 10236-10237.
[63] D. M. H. Newitt, A. E., The formation of methly alcohol and formaldehyde in the slow combustion of methane at high pressure, Proc. R. Soc. London Ser. A. (125) (1932).
[64] D. J. Thomas, R. Willi, A. Baiker, Partial oxidation of methane. The role of surface reactions, Industrial and Engineering Chemistry Research 31 (10) (1992) 2272-2278.
[65] P. S. M. Casey, T.; Foger, K., Selective oxidation of methane to methanol at high pressure, Ind. Eng. Chem. Res. 33 (1994).
[66] J. W. Chun, R. G. Anthony, Catalytic oxidations of methane to methanol, Industrial and Engineering Chemistry Research 32 (2) (1993) 259-263.
[67] C. Hammond, M. M. Forde, M. H. Ab Rahim, A. Thetford, Q. He, R. L. Jenkins, N. Dimitratos, J. A. Lopez-Sanchez, N. F. Dummer, D. M. Murphy, A. F. Carley, S. H. Taylor, D. J. Willock, E. E. Stangland, J. Kang, H. Hagen, C. J. Kiely, G. J. Hutchings, Direct Catalytic Conversion of Methane to Methanol in an Aqueous Medium by using Copper-promoted Fe-ZSM-5, Angew. Chem. Int. Ed. 51 (2012) 1-6.
[68] G. C. Fortman, N.C. Boaz, D. Munz, M. M. Konnick, R. A. Periana, J. T. Groves, T. B. Gunnoe, Selective Monooxidation of Light Alkanes Using Chloride and lodate, J. Am. Chem. Soc. 2014, 136, 8393-8401 136 (2014) 8393-8401.
[69] B. Vora, J. Q. Chen, A. Bozzano, B. Glover, P. Barger, Various routes to methane utilization-SAPO-34 catalysis offers the best option Catalysis Today 141 (1-2) (2009) 77-83.
[70] M. C. Alvarez-Galvan, N. Mota, M. Ojeda, S. Rojas, R. M. Navarro, J. L. G. Fierro, Direct methane conversion routes to chemicals and fuels, Catalysis Today 171 (1) (2011) 15-23. https://doi.org/http://dx.doi.org/10.1016/j.cattod.2011.02.028.
[71] V. S. Arutyunov, Recent results on fast flow gas-phase partial oxidation of lower alkanes, Journal of Natural Gas Chemistry 13 (2004) 10-22.
[72] V. L. Sushkevich, J. A. van Bokhoven, Methane-to-Methanol: Activity Descriptors in Copper-Exchanged Zeolites for the Rational Design of Materials, ACS Catalysis 9 (7) (2019) 6293-6304. https://doi.org/10.1021/acscatal.9b01534.
[73] T. Yu, Z. Li, L. Lin, S. Chu, Y. Su, W. Song, A. Wang, B. M. Weckhuysen, W. Luo, Highly Selective Oxidation of Methane into Methanol over Cu-Promoted Monomeric Fe/ZSM 5, ACS Catal. 11 (2021) 6684-6691.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A device comprising:
a reactor having a plurality of reactor tubes, wherein walls of the reactor tubes are coated with an inert coating, wherein the reactor tube is made of a nickel-based metal alloy.

2. The device of claim 1, wherein the nickel-based metal alloy has a composition comprising: a weight percent of about 58 to 66% Ni, a weight percent of about 20 to 23% Cr, a weight percent of about 8 to 10% Mo, a weight percent of about 0.01 to 5% Fe, a weight percent of about 3 to 4% Nb and Ta, and a weight percent of about of about 0.01 to 1% Co.

3. The device of claim 1, wherein the metal alloy is Inconel® 625.

4. The device of claim 1, wherein the inert coating is a carbon/carbide layer.

5. The device of claim 4, wherein the carbon/carbide layer comprises graphite oxide, semi-amorphous carbon, graphitic carbon, chromium carbide phase Cr₇C₃ and chromium carbide phase Cr₂₃C₆.

6. The device of claim 1, wherein the inert coating is about 50 to 200 microns thick.

7. The device of claim 1, wherein each of the reactor tubes have an inside diameter of about 0.9 to 1.1 millimeters.

8. The device of claim 1, wherein each reactor tube has a mixing zone, a hot zone, and a quench zone, where each reactor tube has a length, wherein the mixing zone is the a first portion of the length of the reactor tube, wherein the hot zone is a second portion of the length of the reactor tube, and the quench zone is a third portion of the length of the reactor tube, wherein the second portion is between the first portion and the third portion, wherein the first portion is adjacent an entrance to the reactor tube and the third portion is adjacent the exit of the reactor tube wherein the first portion is about 8 to 50% of the length, wherein the second portion is about 7 to 20% of the length, wherein the third portion is about 2 to 50% of the length.

9. A system for selectively oxidizing methane to methanol, comprising: a reactor of claim 1, wherein the system has the characteristic of a per pass methanol yield of greater than 8% and selectivities of greater than 70% for methane to methanol.

10. The system of claim 9, wherein the system has a total reactor pressure of about 80 bar during operation, an outer wall temperature of about 380-440° C. in a hot zone of the reactor tube, an outer wall temperature of about 200-250° C. in a quench zone of the reactor tube, and wherein a methane and air mixture having a methane/air molar ratio 2.5 to 3.3 is introduced to the reactor tubes to a pressure of about 70 to 90 bar and the methane and air mixtures has a residence time in the reactor tubes of about 0.8 min.

11. The system of claim 9, wherein a temperature of each zone of the reactor tubes are controlled using a heat bath, a heat jacket, a chiller, or a combination of these,
wherein the system further comprises a gas flow system in gaseous communication with the entrance and the exit of the reactor tubes, wherein the gas flow system is configured to flow a methane and air mixture into the entrance of the reactor tubes, wherein the ratio of the methane to air mixture is controlled using mass flow controllers, wherein a backpressure regulator is in gaseous communication with the exit of the reactor tubes, wherein the backpressure regulator is configured to control the pressure in the reactor, wherein the gas flow system is configured to flow the methane and air mixtures into the reactor tubes to reach a pressure of about 70 to 90 bar, wherein the reactor is configured to control the temperature of the outer wall of the hot zone to be about 380-440° C., wherein the reactor is configured to control the temperature of the outer wall of the quench zone to be about 200-250° C., wherein the gas flow system is configured to receive the methanol from the exit of the reactor tubes resulting from the oxidization of the methane to methanol.

12. A method of selectively oxidizing methane to methanol, comprising:
introducing methane and air to the reactor of claim 1, wherein the methane and air are introduced via the entrance of the reactor tubes at a ratio of about 2.5 to 3.3 until a pressure of 60 to 100 bar is reached;
regulating a temperature of the hot zone of the reactor tubes to be about 380-440° C.;
forming a mixture comprising methanol;
regulating a temperature of the quench zone of the reactor tubes to be about 200-250° C.; and
collecting the mixture comprising the methanol.

13. The method of claim 12, wherein the method has a per pass methanol yield of greater than 8%.

14. The method of claim 12, wherein the method has a selectivities of greater than 70% for methane to methanol.

15. A method of coating a reactor wall, comprising:
introducing propane to a reactor tube of a reactor, wherein the reactor tube is made of a nickel-based metal alloy;
decomposing the propane at a temperature of about 650 to 750° C., and
forming an inert coating on the inside walls of the reactor tube.

16. The method of claim 15, wherein the nickel-based metal alloy has a composition comprising: a weight percent of about 58 to 66% Ni, a weight percent of about 20 to 23% Cr, a weight percent of about 8 to 10% Mo, a weight percent of about 0.01 to 5% Fe, a weight percent of about 3 to 4% Nb and Ta, and a weight percent of about of about 0.01 to 1% Co.

17. The method of claim 15, wherein the metal alloy is Inconel® 625.

18. The method of claim 15, wherein the inert coating is a carbon/carbide layer.

19. The method of claim 15, wherein the carbon/carbide layer comprises graphite oxide, semi-amorphous carbon, graphitic carbon, chromium carbide phase Cr₇C₃ and chromium carbide phase Cr₂₃C₆.

20. The method of claim 15, wherein the inert coating is about 50 to 200 microns thick and wherein each of the reactor tubes have an inside diameter of about 0.9 to 1.1 millimeters.

21. (canceled)
